(19) United States
(12) Patent Application Publication
(10) Pub. No.: US 2015/0173666 A1
(43) Pub. Date: Jun. 25, 2015

(54) In-Situ Concussion Monitor

(71) Applicant: Integrated Bionics, LLC, Richmond, TX (US)

(72) Inventors: Stéphane Louis Smith, Richmond, TX (US); Yves Kevin Smith, Richmond, TX (US)

(21) Appl. No.: 14/578,032

(22) Filed: Dec. 19, 2014

Related U.S. Application Data
(60) Provisional application No. 61/919,378, filed on Dec. 20, 2013.

(51) Int. Cl. A61B 5/00; A61B 5/01; A61B 5/16; A61B 5/0476; A61B 5/0402; A61B 5/11; A61B 5/145

(52) U.S. Cl. CPC A61B 5/4064; A61B 5/1118; A61B 5/01; A61B 5/14542; A61B 5/0476; A61B 5/0402; A61B 5/1112; A61B 5/1121; A61B 5/162; A61B 5/4023; A61B 5/4875; A61B 5/6802; A61B 5/7405; A61B 5/742; A61B 5/7455

(57) ABSTRACT

An in-situ physiologic monitor for a body is disclosed. The in-situ physiologic monitor comprises an electronic monitoring module, wherein the electronic monitoring module is capable of logging an electronic monitoring module sensed parameter; and a body attachment component, wherein the body attachment component attaches the electronic monitoring module to the body.

### CROSS REFERENCE TO RELATED APPLICATIONS

This application is a non-provisional of U.S. Provisional Patent Application Ser. No. 61/919,378, entitled “An In-Situ Concussion Monitor,” filed on Dec. 20, 2013, the entire disclosure of which is incorporated herein by reference.

### BACKGROUND

Embodiments relate to power-efficient on-field health and performance management monitors, and in certain embodiments, to in-situ physiologic monitors which may provide on-field monitoring of health and athletic performance while consuming minimum amounts of power.

Traumatic head injuries (THI), which may include concussive and sub-concussive hits, have been correlated with long term chronic and deleterious effects on the brain. Cumulative effects of THI may result in psychiatric disorders and long term memory loss. Concern exists that repetitive head injuries may impact brain development in children and adolescence.

Despite attempts to improve athletic equipment to mitigate THI, such injuries remain a significant problem. Some research has even demonstrated that existing head protection equipment may actually increase head accelerations and exacerbate the risk of concussions. To properly manage THI, it may be necessary to remove athletes suspected of sustaining a concussion from play. Athletes who are not removed, or who are returned to play prematurely after sustaining a concussive event may be at an increased risk of a catastrophic outcome known as second impact syndrome. Second-impact syndrome (SIS) may occur when the brain swells rapidly, and catastrophically, after a person suffers a second concussion before symptoms from an earlier concussion have subsided. The second concussion may occur minutes, days, or weeks after the prior concussion. Even mild grades of concussions may lead to SIS. SIS is often fatal, and even the survivors may be severely disabled.

Therefore the accurate detection and monitoring of concussive events is of the utmost importance in athletic events where such concussive events may occur. Such detection and monitoring may be even more difficult in athletic events which do not use helmets or other types of protective headgear.

### SUMMARY

An embodiment comprises an in-situ physiologic monitor for a body. The in-situ physiologic monitor comprises an electronic monitoring module, wherein the electronic monitoring module is capable of logging an electronic monitoring module sensed parameter; and a body attachment component, wherein the body attachment component attaches the electronic monitoring module to the body.

An embodiment comprises a method of conserving power in an in-situ physiologic monitor for a body. The method comprises providing an in-situ physiologic monitor comprising: an electronic monitoring module; wherein the electronic monitoring module is capable of logging an electronic monitoring module sensed parameter; wherein the electronic monitoring module comprises a low fidelity sensor and a high fidelity sensor; wherein the low fidelity sensor consumes less power than the high fidelity sensor; and wherein the high fidelity sensor is capable of sensing in a higher fidelity than the low fidelity sensor; and a body attachment component, wherein the body attachment component attaches the electronic monitoring module to the body. The method further comprises using the low fidelity sensor to induce sensing with the high fidelity sensor.

### BRIEF DESCRIPTION OF THE DRAWINGS

The following figures are part of the present specification, included to demonstrate certain aspects of embodiments of the present disclosure and referenced in the detailed description herein. Unless otherwise noted, figures are not drawn to scale.

FIG. 1 is an illustration of an in-situ physiologic monitor comprising a body attachment component and electronic monitoring module in accordance with certain embodiments.

FIG. 2 is another illustration of an in-situ physiologic monitor comprising a body attachment component and electronic monitoring module in accordance with certain embodiments.

FIG. 3 is another illustration of an in-situ physiologic monitor comprising a body attachment component and electronic monitoring module in accordance with certain embodiments.

FIG. 4 is another illustration of an in-situ physiologic monitor comprising a body attachment component and electronic monitoring module in accordance with certain embodiments.

FIG. 5 is another illustration of an in-situ physiologic monitor comprising a body attachment component and electronic monitoring module in accordance with certain embodiments.

FIG. 6 is an illustration of an in-situ physiologic monitor comprising a body attachment component and electronic monitoring module in accordance with certain embodiments.

FIG. 7 is an exploded view of an electronic monitoring module in accordance with certain embodiments.

FIG. 8 is an illustration of an electronic monitoring module in accordance with certain embodiments.

FIG. 9 is an illustration of a finite state machine computational model for use with the in-situ physiologic monitor in accordance with certain embodiments.

### DETAILED DESCRIPTION

Embodiments relate to power-efficient on-field health and performance management monitors, and in certain embodiments, to in-situ physiologic monitors which may provide on-field monitoring of health and athletic performance while consuming minimum amounts of power.

A critical problem for on-field human health and performance management is that the monitors used to measure health and performance may require a trade-off between the overall longevity of a said monitor and the data fidelity that it is capable of capturing. Monitors generally comprise various different types of sensors to measure and record health and performance data. These sensors may possess differing capabilities and may generally be categorized as either high fidelity or low fidelity. High fidelity sensors may consume significantly more power than their lower fidelity counterparts, but in turn, may feature faster sampling rates, increased resolution, increased dynamic range, improved precision, improved noise tolerance, as well as other metrics. Low fidelity sensors may exhibit extended longevity but poor data fidelity (e.g., limited dynamic range, resolution, noise, etc.) In the traditional approach, sensor selection is made based on a required specification, forcing trade-off between data fidelity and device longevity.

The examples disclosed herein disclose apparatuses, methods, and systems for on-field health and performance management that high fidelity sensors capable of extended use. The disclosure illustrates the combination of at least some of the aspects of both low fidelity and high fidelity sensors. Further, embodiments described herein illustrate the management of said sensors so as to achieve optimal data gathering efficiency.

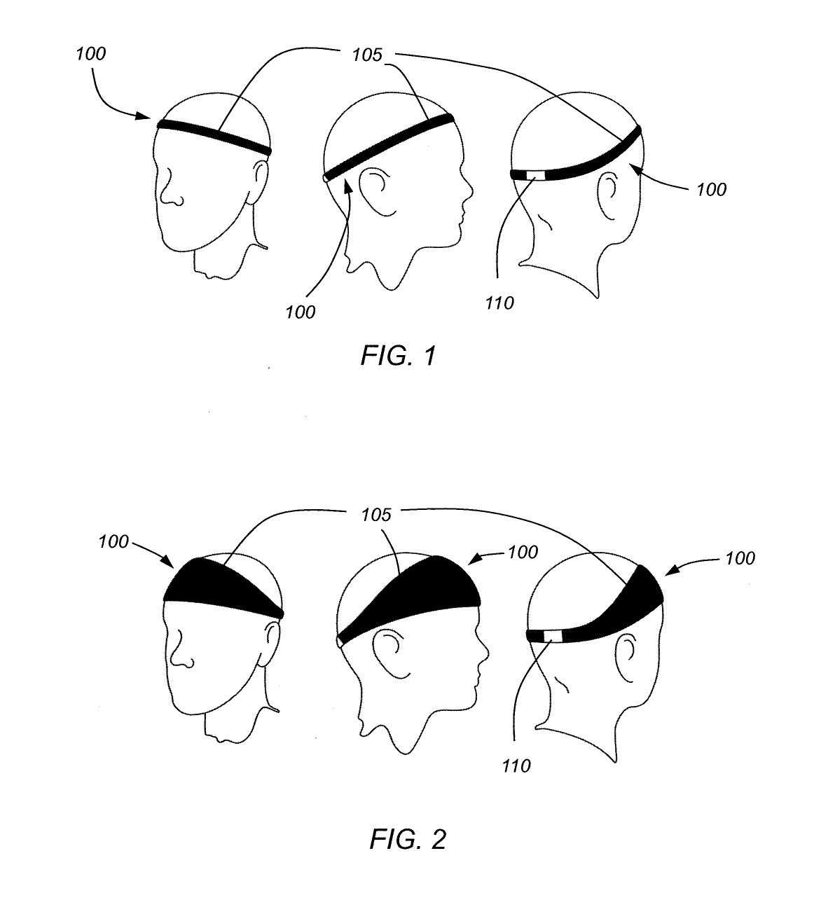

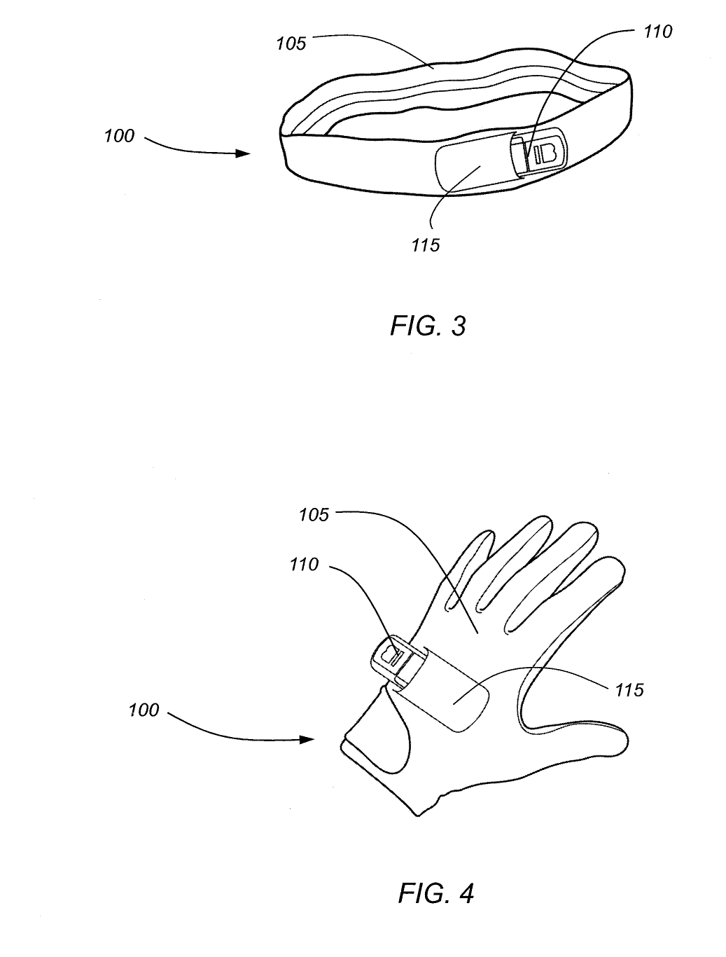

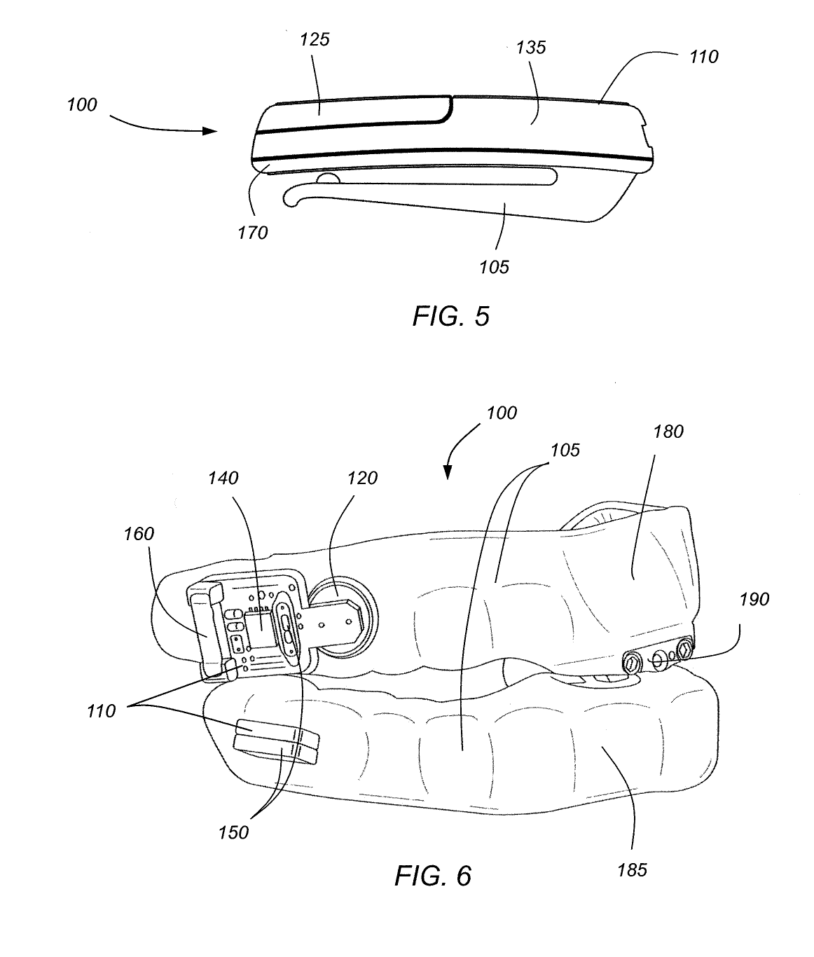

With reference to FIGS. 1-6, various embodiments of an in-situ physiologic monitor (ISPM) 100 are illustrated. The in-situ physiologic monitor 100 may comprise an electronic monitoring module (EMM) 110 and a body attachment component (BAC) 105. The EMM 110 may be configured to capture data relevant to the health and performance of the wearer. The BAC 105 may be designed to attach the EMM 110 directly to the wearer's body, including head, hands, feet, legs, arms, shoulders, torso, teeth, jaw, and the like. The BAC 105 may comprise a headband (as illustrated by FIGS. 1 and 2), gloves (as illustrated by FIG. 4), clothing, clip (as illustrated by FIG. 5), oral appliance (as illustrated by FIG. 6), helmet, or any such means sufficient for attachment of the EMM 110 to a body. EMM 110 may be attached to the BAC 105 in any sufficient manner. For example, in some embodiments, BAC 105 may additionally comprise an enclosure 115 (as illustrated in FIGS. 3 and 4) to contain an EMM 110 if desired. In alternative embodiments, BAC 105 may comprise an integrated EMM 110. In still further embodiments, EMM 110 may comprise a clip (as illustrated in FIG. 5) to clip onto an article of clothing or athletic equipment worn by the ISPM 100 user or alternatively to a BAC 105 worn by the ISPM 100 user. In other embodiments, the EMM 110 may be affixed to a BAC 105 comprising an oral appliance (as illustrated in FIG. 6). In embodiments, the ISPM 100 may be configured to be worn on the body during an athletic activity, as well as any other activity. “Athletic activity” as used herein, does not refer to a specific sport or athletic event, but instead merely refers to any activity in which the wearer of the ISPM 100 may potentially sustain a THI and/or desires to quantify and track their health and performance metrics. The ISPM 100 may promote extended use by the user by minimizing the amount of required interactions for a user, for example, in some embodiments, ISPM 100 may not have an “on/off” or “power” interface. In still further example embodiment, ISPM 100 may not require charging, for example, because ISPM 100 may be configured to maximize battery longevity. Thus, ISPM 100 may incentivize extended usage by reducing the attention needed to maintain ISPM 100 in a functional capacity. As such, ISPM 100 may provide high fidelity human health and performance data without interfering with a user's athletic activity or routine.

In some embodiments, ISPM 100 may be configured to record the biometric data of a user prior to, during, or subsequent to engagement in an athletic activity. The recorded biometric data may be used to analyze a user's health and performance, and may also be used to track these same health and performance metrics over time. This may aid in the recognition of injuries, which may be too subtle or difficult to detect without performance metric tracking. Recorded biometric data may provide for an objective means of tracking health and performance trends and recognizing health events. A potential THI may trigger the ISPM 100 to report the potential THI. Without limitation, examples of injurious events which may trigger the ISPM 100 may include concussions which occur as a result of repeated head accelerations, concussions which occur as a result of a single large head acceleration, or a combination of both.

With continued reference to FIGS. 1-6, the BAC 105 may be designed to attach the EMM 110 to the wearer's body, for example, BAC 105 may attach the EMM 110 to a wearer's head as illustrated in FIGS. 1 and 2. The BAC 105 may be configured to attach the EMM 110 to the wearer in such a way that the any acceleration, rotation, displacement, alteration of position, etc. of the wearer at the point of attachment are mimicked in the acceleration, rotation, displacement, alteration of position, etc. of the EMM 110. The EMM 110 may log any such EMM sensed parameters. An EMM sensed parameter may be any such parameter capable of being sensed by one or more sensors of the EMM. Without limitation, the EMM sensed parameters may comprise acceleration, rotation, displacement, and/or position information as a proxy for any acceleration, rotation, displacement, and/or position information experienced by the wearer; additional health and performance data (e.g., heart rate, temperature, distance, time, calories burned, speed, sprint acceleration, hydration, balance, reaction time, etc.) may be logged if desired. The recorded information may be used to determine the possible occurrence of a specific event (e.g., a THI). As discussed above, the BAC 105 may take numerous forms including but not limited to headbands, skullcaps, helmets, goggles, facemasks, hair ties, swim caps, shin guards, elbow pads, knee pads, gloves, wristbands, anklebands, shoulder pads, shoes, socks, ice skates, bras, jerseys, oral appliances, clips, cheer bows, and chest straps. In some embodiments, headbands used to control hair and sweat during play and may function as the BAC 105 by providing an EMM 110. Internal silicone lining may provide resistance to headband slippage. All styles of headbands including constant width as well as tapering are among possibilities. Narrow headbands, as illustrated by FIG. 1 may be used to provide an EMM 110 with minimal contact to the wearer. Conversely, wider headbands as illustrated by FIG. 2 may be used to provide additional sweat and hair control as well as provide reduced slippage. In other embodiments, hair ties may function as the BAC 105 and may be made to look like conventional hair ties, while also providing an EMM 110. FIG. 3 illustrates an EMM 101 disposed within an enclosure 115 on a BAC 105. The BAC 105 illustrated in FIG. 3 may be a headband, wristband, ankleband, chestband, etc. FIG. 4 illustrates an EMM 110 disposed within an enclosure 115. The BAC 105 illustrated in FIG. 4 is a glove which may be worn by the user to track the acceleration of and impacts to the hand. The BAC 105 may be any shape or have any dimension necessary to provide a sufficient attachment of EMM 110 to a desired location of the wearer. Further BAC 105 may comprise any means of attachment of EMM 110 to the BAC 105 and consequently to the wearer. Such means may include, but should not be limited to clips (as illustrated by BAC 105 comprising a clip in FIG. 5), clasps, hooks, fasteners, pockets, adhesive etc. FIG. 6 illustrates a BAC 105 comprising an oral appliance. The oral appliance BAC 105 may be placed over the wearer's teeth. The oral appliance BAC 105 may be suitable for applications in which it may not be practical to attach an EMM 110 to a BAC 105 comprising a headband. BAC 105 may be made of any such material sufficient for its purpose including rubber, plastics, acrylics, fabrics, leathers, metals, etc. The material chosen may be dependent upon the desired application. For example a BAC 105 comprising an oral appliance may be produced from a different material than a BAC 105 comprising a headband.

In embodiments, the EMM 110 may be disposed about or inside a BAC 105. In some embodiments, the BAC 105 may be permanently joined with the EMM 110 such that the BAC 105 and the EMM 110 may not separate. For example, an EMM 110 may be sewn into the BAC 105, glued onto the BAC 105, or be designed and integrated as a single unit. In another example, the EMM 110 may be permanently entombed and hermetically sealed within an oral appliance as seen in FIG. 6. In other embodiments, the BAC 105 and EMM 110 may be configured to be separated as desired. In these embodiments, the EMM 110 may be disposed within an enclosure 115 in the BAC 105 as illustrated in FIGS. 3 and 4. FIG. 5 illustrates a, BAC 105 comprising a clip which may function by clipping the EMM 110 across a headband, clipping inside of a helmet, clipping to a glove, clipping to a belt, clipping to a shin guard, clipping to any other piece of clothing or equipment worn, or clipping directly to the user should the clip be sufficient for doing so. In these embodiments, BAC 105 may be removable from EMM 110, separating the clip from the EMM 110 and isolating the EMM 110 (as illustrated in FIG. 8). Other options include the EMM 110 being wrapped into the BAC 105, attached via fasteners to the BAC 105, attached via an adhesive to the BAC 105, held in place via undercuts designed in the BAC 105, or any other method of reliably securing the EMM 110 about or to the BAC 105. In applications with output devices comprising visual indicators, if an enclosure 115 is used, the enclosure 115 may be made of a mesh, translucent, or transparent material to enable transmission of visual system events and alerts through the BAC 105. The EMM 110 may be removed for numerous purposes including replacing a battery, updating firmware, downloading logged data, exchanging a BAC 105 as desired for a different BAC 105, or for any other purpose.

In some embodiments, the EMM 110 may be hermetically sealed within a housing. Benefits of hermetically sealing the EMM 110 may include protecting the EMM's 110 electronics, enabling EMM 110 to safely interface with the human body (e.g., inside the mouth), or enabling the EMM 110 to be machine washable with or without the BAC 105. This may also make the EMM 110 biocompatible and/or minimize the user's ability to access the EMM's 110 electronics. In alternative embodiments, the EMM 110 may not be heimetically sealed. In these embodiments, the EMM 110's electronics may be accessible for numerous purposes such as enabling tactile buttons, ports for audio input and output, battery charging ports, the replacement of a battery, updates to the firmware, the downloading of logged data, or for any other purpose as would occur to one skilled in the art.

In some embodiments, the EMM 110 may be used to detect head, body, or extremity tilt. An example may include using the EMM 110 in situ to correct head, body, or extremity position.

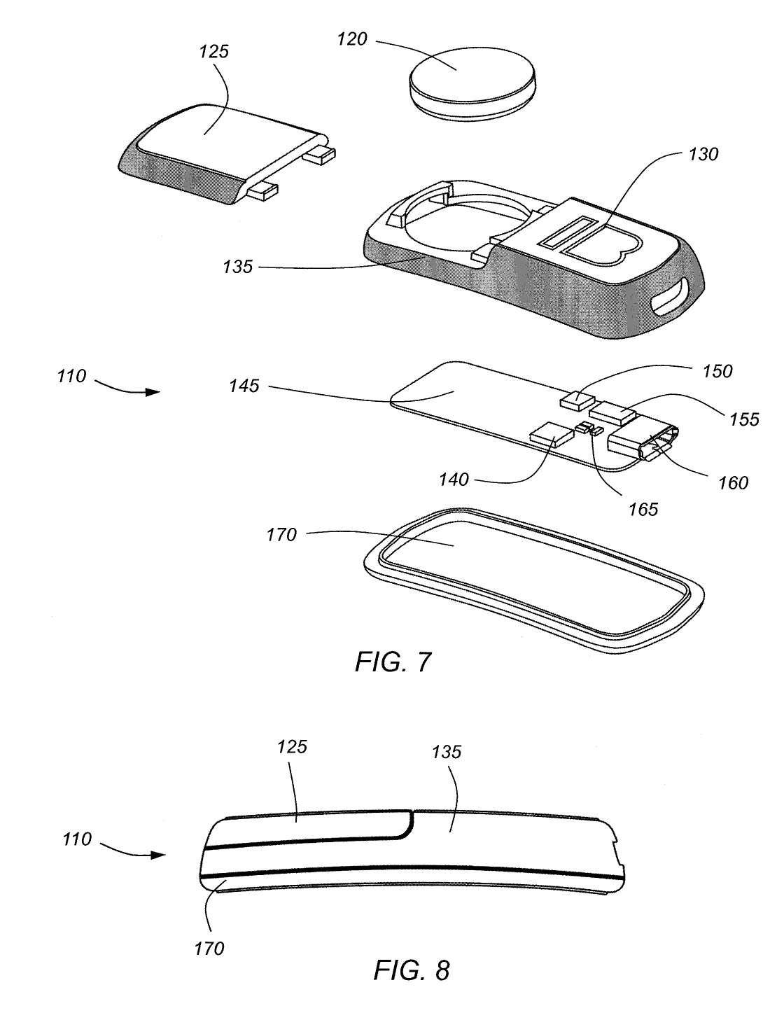

With reference to FIG. 6, the ISPM 100 may comprise a BAC 105 which comprises an oral appliance. An oral appliance may function as an athletic mouth guard or in other embodiments such as a mandibular repositioning device (MRD). An MRD may comprise a maxillary component 180, a mandibular component 185, and a mechanism 190 for holding the maxillary and mandibular component in such a way that the mandible is protruded relative to the maxilla. The EMM 110 may comprise a battery 120, processor 140, low fidelity sensor 150, high fidelity sensor (embedded in processor 140), and a bi-directional communication module 160 which may be analogous to the components descriptions described above and also below with reference to FIG. 7. The high fidelity sensor may be a temperature sensor directly embedded in processor 140. The low fidelity sensor 150 may comprise a magnet and a magnetic reed relay. The low fidelity sensor 150 components may be disposed about BAC 105, such that the magnet may be disposed on the mandibular component 185 and the reed relay may be disposed on the maxillary component 180. The bi-directional communication module 160 may comprise an RFID antenna and communication module. The EMM 110 may be hermetically sealed and permanently fixed to BAC 105.

With reference to FIG. 7, the EMM 110 may comprise a battery 120, battery door 125, top housing 135, window 130, circuit board 145, processor 140, a low fidelity sensor 150, a high fidelity sensor 155, a bi-directional communication module 160, an output device 165, and bottom housing 170. It is to be understood that the above components are optional, and the EMM 110 may be used with more than or less than the components described above. For example, the EMM 110 may be used without any top housing 135 or bottom housing 170 if desired. Further, in some embodiments, bi-directional communication module 160 may fulfill one or more functions of the input device(s) (not shown) and/or output device(s) 165. The EMM 110 may comprise a plurality of low fidelity sensors 150 and/or high fidelity sensors 155. Without limitation, low fidelity sensors 150 may comprise accelerometers, magnetic sensors, reed relays, RFID, photo-sensors, pressure sensor, piezoelectric sensors, tilt sensors, microphones, radio antennas, proximity sensor, tactile sensor, or any combination thereof. Without limitation, high fidelity sensors 155 may comprise thermocouples, pressure sensors, accelerometers, electro-chemical sensors, gyroscopes, humidity sensors, microphones, acoustic sensor, vibration sensors, temperature sensors, hydration sensors, humidity sensor, moisture sensor, proximity sensor, light sensor, tilt sensor, inertial measurement unit sensor, compass, inclinometer, altimeter, GPS, pulse-oximeter sensors, EEGs, EKGs, voltmeters, ammeters, capacitance meter, inductance meter, resistance meter, LCR meter, or any combination thereof. Further examples of low fidelity sensors 150, described as companion sensors, and high fidelity sensors 155, described as primary sensors, may be found in U.S. patent application Ser. No. 13/934,432 the disclosure of which is incorporated herein by reference. Bottom housing 170 may positionally secure circuit board 145 within top housing 135. EMM 110 may comprise any number of input devices, output devices 165, and/or bi-directional communication modules 160. Processor 140 may maintain a system time base by using an oscillator built into processor 140, an external crystal oscillator (not shown), or any other method of as would occur to one skilled in the art.

In embodiments, the low fidelity sensor 150 and the high fidelity sensor 155 may be communicatively coupled such that the low fidelity sensor 150 is able to transition the high fidelity sensor 155 to an active state and vice versa. This may allow the ISPM 100 to conserve power by using the low fidelity sensor 150 to modulate the activity of the high fidelity sensor 155. For example, the high fidelity sensor 155 may remain in a low power setting (e.g., a setting in which it is not actively sampling), a powered off setting, or a sleep/idle setting; until the low fidelity sensor 150 reconfigures or triggers the high fidelity sensor 155 to begin sampling, in which case it may begin to use as much power as necessary to obtain the required fidelity for an application. Using one sensor type to modulate the other allows for a precise use of high fidelity data measurement only when an event requiring such high fidelity data measurement is present. Should no event be present, the sensors may work in concert to reduce power consumption to the minimum level needed to detect the presence of the aforementioned event.

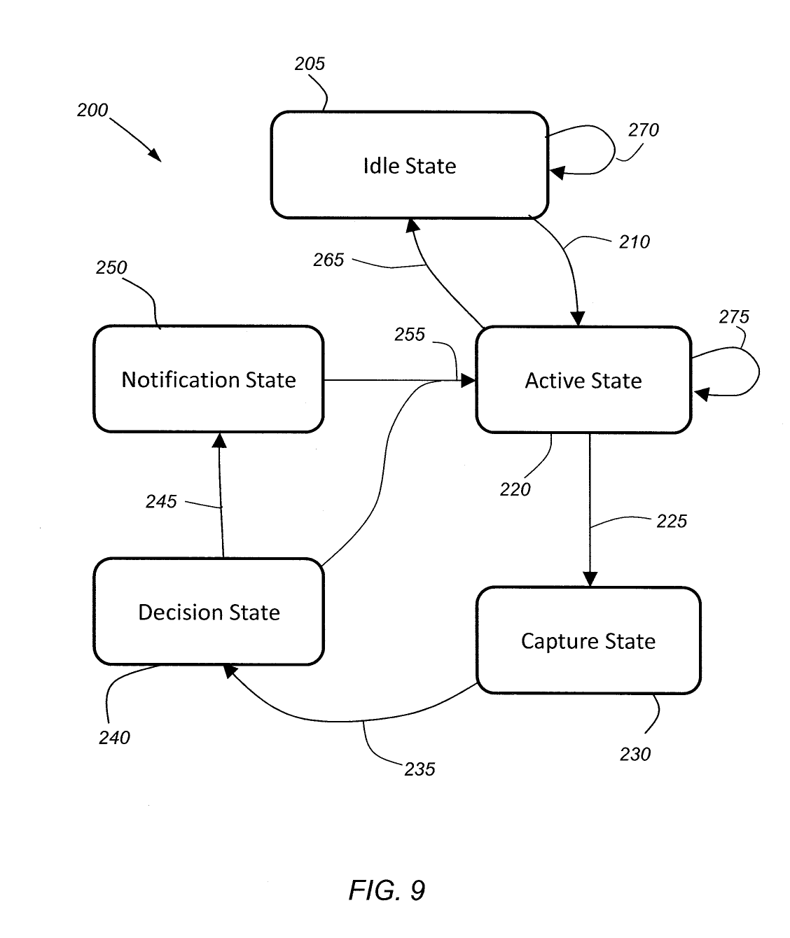

Embodiments of the EMM 110 may comprise a high fidelity sensor 155. The high fidelity sensor 155 may be chosen based on the data requirements during a data capture state (e.g., capture state 230 illustrated in FIG. 9) and the overall power requirements of the ISPM 100. Such requirements may include dynamic range, resolution, bandwidth, sampling rate, power modes, reconfiguration time, and sleep current consumption. In part, the present invention greatly reduces the EMM's overall current consumption by limiting the amount of time the high fidelity sensor 155 operates in its high fidelity measurement mode (the highest power mode). For potential THI recognition, a dynamic range of +200 gs and 800 Hz sampling rate may be desired. Additionally, the high fidelity sensor 155 may be chosen to be quickly reconfigured, as in to power-on quickly or to quickly transition between sleep/idle power mode to active mode. How quickly the high fidelity sensor 155 may be reconfigured or transition to active mode is specific to sensor type, and the required speed for reconfiguration or transition to active mode is specific to the activity with which the ISPM 100 is to be used. For example, a rapid head acceleration above a threshold may result in a sensor (either a low fidelity sensor 150 or high fidelity sensor 155) detecting a capture trigger (as described below and illustrated in FIG. 9) and a transition from active state 220 to capture state 230. During the transition, the EMM 110 may reconfigure the high fidelity sensor 155 into a measurement mode sufficiently fast enough to not miss the target event, e.g., a rapid head acceleration profile. In this specific example, a head impact may take 5 ms to reach peak acceleration, and have a total duration of 20 ms. Additionally, a delay of 1 ms may be incurred for the active state sensor (either a low fidelity sensor 150 or high fidelity sensor 155 dependent upon the active state configuration) to detect the capture trigger. In this example, a high fidelity sensor 155 must be reconfigured or transitioned to active mode in less than 4 ms to measure the peak acceleration during the capture state. The faster the high fidelity sensor 155 is capable of being reconfigured or transitioned to its active mode, the greater the amount of the acceleration profile may be captured. Continuing with the above example, for a high fidelity sensor 155 that can reconfigure in 0.5 ms, the first 1.5 ms of the acceleration profile may be missed, however, the peak acceleration and the remainder of the acceleration profile may be sufficiently captured in the desired application. As discussed above, a plurality of high fidelity sensors 155 may be used in embodiment. The number and type of high fidelity sensors 155 may be dictated by the current application. For example, some embodiments may comprise gyros to determine angular accelerations, velocity, or position, whereas some embodiments may comprise additional accelerometers. In some embodiments, multiple accelerometers may be used in conjunction to determine yaw, pitch, and roll angular accelerations.

Embodiments of the EMM 110 may comprise a low fidelity sensor 150. The low fidelity sensor 150 may be chosen based on the data requirements during the idle state (e.g., idle state 205 illustrated in FIG. 9), the power requirements during idle state, and the overall power requirements of the ISPM 100. Such requirements may include dynamic range, resolution, bandwidth, sampling rate, power modes, reconfiguration time, sleep current consumption, and active current consumption. In part, the present invention greatly reduces the EMM's overall current consumption by maximizing the amount of time the low fidelity sensor 150 operates in its low power measurement mode (the lowest power mode). By selecting a very low power low fidelity sensor 150, the EMM 110 may operate for extended periods of time. For example, a low fidelity sensor 150 current consumption may be only 4 uA in the idle state. A charge storage device such as a battery 120 may comprise a capacity of 100 mAh. Therefore, the EMM 110 may operate in the idle state for 25,000 hours or almost 3 years. In this mode of operation, the EMM 110 may not need a traditional ‘on/off’ button, improving user compliance. In low power mode, the low fidelity sensor 150 must be able to detect an activity trigger, (e.g., walking, running, jumping, etc.). In some embodiments, the low fidelity sensor may comprise a passive sensor (e.g., a magnetic reed relay detecting a magnetic field or RFID antenna detecting an RF field). In these cases, the low fidelity sensor 150 may not have an explicit sampling rate, but nevertheless generates a signal that may be used as the activity trigger.

With continued reference to FIG. 7, embodiments of the EMM 110 may comprise input devices (not shown). Input devices may comprise tactile buttons, microphone, tilt sensor, RFID, or any other input device as would occur to one skilled in the art. An input device may also comprise processed sensor data. For example, an accelerometer may be configured to recognize various gestures. Without limitation, examples of other detectable acceleration gestures include gestures such as a double-tap signature (e.g., as expected from using a conventional computer mouse), a heading signature detected as a player heads a ball, a tilt signature detecting someone on the ground, a free-fall signature detected from a fall. Many gestures may be used concurrently to detect the initiation of different events. A successful detection of a signature may be interpreted by the EMM 110 as an input device. Input devices may be used to turn the EMM 110 on or off, take a data point, self-test, calibrate, modulate sampling rates, enable a bi-directional communication module 160, or any other function that may be required by the target application.

Embodiments of the EMM 110 may comprise output devices 165. Output devices 165 may be used to indicate that a system event and/or an alert has occurred. System events may comprise information related to the EMM 110 including, without limitation, battery status, wired communication status, wireless communication status, system errors, or any other indication that may occur to one skilled in the art. Alerts may comprise information related to the wearer of immediate importance including, without limitation, an acceleration beyond a specified threshold, free-fall acceleration, detection of a possible THI, and exceeding a certain number of head hits. In some embodiments, output device 165 may comprise visual feedback, for example, a light emitting diode (LED) may be used to indicate a possible THI. Visual feedback may vary in color and pulse pattern depending on the system event or health alert being conveyed. Other embodiments of output devices 165 may comprise haptic feedback, tactile feedback (e.g., a vibration actuator), or audible feedback. Yet still other embodiments may transmit via an RF communication module. A plurality of output devices 165 may be included as part of the EMM 110.

In some embodiments, bi-directional communication module 160 may be used to communicate with the EMM 110. The bi-directional communication module 160 may be used to enable the EMM 110 to transmit recorded data, transmit system data, receive update settings, receive firmware updates, or receive commands to execute. In some embodiments, the bi-directional communication module 160 may perform all functions of an input device, and may perform all functions of an output device 165 such as transmit system events and alerts. Without limitation, bi-directional communication module 160 may include wire interfaces, USB, UART, BlueTooth, BlueTooth Low-Energy, Wifi, RFID, proprietary RF protocol, infrared communication, optical communication, voice/speaker communication, and others.

With continued reference to FIG. 7, in some embodiments, the processor 140 may be configured to store collected measurements from at least one low fidelity sensor 150 and/or a high fidelity sensor 155 as information on memory (not shown). Without limitation, memory may comprise read-only memory, random-access memory, or the like. The memory may be external or internal as desired. In embodiments, comprising memory, the memory may be communicatively coupled to processor 140. In alternative embodiments comprising a bi-directional communication module 160, the EMM may transmit data via the bi-directional communication module 160 instead of, or in addition to, recording the measurements on memory. The bi-directional communication module 160 may couple the EMM 110 to an external system. Communication may be done via direct connection, wired connection, a private network, a virtual private network, a local area network, a wide area network (“WAN”), a wireless communication system, or combinations thereof. In some alternative embodiments, the data may not be recorded on memory, or as discussed above, memory may not be used. In some embodiments, the data may be processed in real-time by the processor 140. Processor 140 may generate system events or health alerts using at least one of the EMM 110's output devices 165 and/or bi-directional communication module 160.

Embodiments of the EMM 110 may comprise a battery 120 or other charge storage device. The battery may be configured to be removable such that it is capable of being replaced. In such embodiments, a removable battery door 125 may allow access to a battery compartment. The battery door 125 may directly latch and lock to the top housing 135 or bottom housing 170. In alternative embodiments, the battery may be a fixed component of the EMM 110.

Embodiments of the EMM 110 may comprise graphic art or logoing treatment. Graphic art may be applied to battery door 125, top housing 135, and/or bottom housing 170 to display brands, logos, and provide visual appeal.

In some embodiments, battery door 125, top housing 135, and bottom housing 170 may comprise a soft or rubberized shell or bumper. The battery door 125, top housing 135, and bottom housing 170 may be completely or partially made of a rubberized material. In some embodiments, a soft or rubberized bumper may encircle the top housing 135, battery door 125, and/or bottom housing 170. In these embodiments, the soft or rubberized shell or bumper may be used to promote the user's comfort and safety.

In some embodiments, battery door 125, top housing 135, and bottom housing 170 may comprise a writable surface. The writable surface may also function as an exterior configured for permanent or removable labels which may be uniquely identifiable or written on. For example, an athletic trainer may manage multiple ISPM's 100 and desire to uniquely identify each individual EMMs 110 via writable surface and/or the aforementioned labels.

Embodiments of the EMM 110 may enable input devices and output devices 165 to transmit through the battery door 125, top housing 135, and/or bottom housing 170. For example, when visual indicator output devices 165 are used, for example an LED, a fully or partially translucent window 130 may be used to pass light through the top housing 135. Window 130 may be stylized and take the form of a logo or any other shape. In other examples, the EMM 110 may incorporate holes to allow the transmission of sound into and out of the EMM 110 housing 135 for an input device (e.g., a microphone) or output device (e.g., a speaker).

With reference to FIG. 8, the profile view of an embodiment of an EMM 110 is displayed. FIG. 8 depicts how a battery door 125, top housing 135, and bottom housing 170 may engage together. Additionally, FIG. 8 shows a particular embodiment where battery door 125, housing 135, and bottom housing 170 are curved to better conform and fit to a user's body. For example, when EMM 110 is worn on the head, such curvature may promote user comfort and safety. The embodiment of FIG. 8 may also be attached to a BAC 105 (not shown) in several ways, for example by using an enclosure 115 (as illustrated in FIGS. 3 and 4) or by using a BAC 105 comprising a clip (as illustrated in FIG. 5).

Now referring to FIG. 9, the EMM 110 may comprise an internal finite state machine 200 (“FSM”) to sequence states and manage the power consumption of the EMM 110 components. As illustrated in FIG. 9, example FSM 200 describes an implementation of some embodiments used for athlete health and performance management. These embodiments may be used to aid in the recognition of THI. The FSM 200 may comprise the following states: idle state 205, active state 220, capture state 230, decision state 240, and notification state 250. Once powered on, the EMM 110 may begin in the idle state 205.

With continued reference to FIG. 9, in some applications, when the ISPM 100 is not in use, the ISPM 100 may be physically motionless. While motionless, the FSM 200 remains in the idle state 205. During the idle state 205, the low fidelity sensor 150 is enabled and the high fidelity sensor 155 is not enabled. In the idle state 205, the low fidelity sensor 150 may consume less power relative to a low fidelity sensor 150 in the active state 220 and also relative to a high fidelity sensor 155 in the idle state 205 or in the active state 220. The EMM 110 may be configured to be in the idle state 205 as much as is reasonable for sufficient functionality in order to prolong the operational longevity of the EMM 110. Thus, a low fidelity sensor 150 comprising a low rate of power consumption may be important to provide an EMM 110 with sufficient operational longevity. In the idle state 205, the low fidelity sensor 150 may continuously poll to detect an activity trigger. The activity trigger may be an activity in which a sufficient amount of movement (e.g., an acceleration above specific threshold induced from walking, running, jumping, etc.) triggers the transition to the active state 220. The activity trigger is defined by the activity in which the ISPM 100 is utilized. For example, a threshold may be preprogrammed based on the needs of the user for the activity in which the ISPM 100 will be engaged. In a specific embodiment, an acceleration in excess of a ±1 g sampled rate of 25 Hz may be set as the activity trigger for a walking activity (resulting in a low current consumption of 4 uA). A detected activity trigger induces transition of the FSM 200 to the active state, via active state transition 210. Active state transition 210 is a transitional period in which the FSM 200 may alter sensor configurations, throttle sensor fidelity rates up, collect new and/or different data, begin polling for new triggers, and/or generally perform new tasks in addition to or to the exclusion of the tasks performed in idle state 205.

Further, tasks may be performed while in the idle state 205. For example, the EMM 110 may also detect and handle input and output operations, periodically wake to sparsely take data points, or perform any other suitable idle state task 270 as desired or required by an application. In one embodiment, data points may be spaced many hours apart when there is little information to gather. Upon completion of any idle state task 270, the FSM 200 may return back to idle state 205.

With continued reference to FIG. 9, the active state 220 defines the period when the EMM 110 actively samples to detect a capture trigger. The FSM 200 transitions to the active state 220 from the idle state 205 when the low fidelity sensor 150 detects an activity trigger 210 as described above. During the active state 220, different sensor configurations are possible with the primary requirement being the sensors must be configured to be able to detect a capture trigger. The capture trigger defines an event that necessitates measuring data at high fidelity in the capture state 230 such that said data may be processed in the decision state 240 and a potential health alert generated in the notification state 250. For example, it may desirable to recognize THI due to a head impact and consequently generate an alert. In a specific example, a capture trigger may be a rapid head acceleration in excess of ±15 gs. Exceeding a programmable sensor reading above a certain threshold, as illustrated by the rapid head acceleration example above, indicates the need to capture data at high fidelity settings such that said data may be processed to generate an alert if needed. Once the capture trigger has occurred, high fidelity data recording may be induced, such that the sensor data subsequent to the capture trigger is recorded in high fidelity. The exact parameters of the capture trigger are defined by the application in which the ISPM 100 is utilized. Thus a capture trigger may be a tilt of a certain degree, an acceleration above a target threshold, etc. As an example, the capture trigger may be different for a boxing application relative to a track and field application. Further, the capture trigger may vary on an individual basis. For example, a capture trigger for football may be head acceleration, but the capture trigger threshold may be different for youth football as compared to professional football, or by gender, age, height, weight, and position.

Continuing with FIG. 9, the active state 220 may comprise a variety of different sensor configurations. One of the potential active state 220 sensor configuration embodiments comprises the low fidelity sensor 150 being reconfigured to detect a capture trigger by throttling up the low fidelity sensor 150 data rate and resolution to a fidelity necessary to capture the capture trigger. In this specific embodiment, the high fidelity sensor 155 remains disabled. For example, if in the idle state 220, the low fidelity sensor 150 had been configured at ±1 g and a sampling rate of 25 Hz, this same low fidelity sensor 150 may be reconfigured to detect a capture trigger (e.g., resulting from a rapid head movement) with an acceleration in excess of ±15 gs at a sampling rate of 400 Hz while using possessing a power consumption of 36 uA. In this specific example, the high fidelity sensor 155 remains in its low power state (not sampling) or is disabled. In an alternative embodiment, the high fidelity sensor 155 may be enabled to detect the capture trigger. For example, the high fidelity sensor 155 may be configured to detect the capture trigger with accelerations of ±15 gs at a sampling rate of 400 Hz and current consumption of 90 uA. In this embodiment, the low fidelity sensor 150 may remain enabled to continue to capture data during an activity (e.g., walking), or alternatively, the low fidelity sensor 150 may be completely disabled to minimize the overall active state 220 power consumption. Other configurations are possible, which, with the disclosure provided herein, will be readily apparent to one having ordinary skill in the art. When choosing which configuration to use, one having ordinary skill in the art may consider choosing the configuration that provides the smallest power consumption that still enables the detection of the capture trigger. When the capture trigger has been detected, the FSM 200 transitions from the active state 220 to the capture state 230. Thus, a detected capture trigger induces transition of the FSM 200 to the active state, via capture state transition 225. Capture state transition 225 is a transitional period in which the FSM 200 may alter sensor configurations, throttle sensor fidelity rates up, collect new and/or different data, begin polling for new triggers, and/or generally perform new tasks in addition to or to the exclusion of the tasks performed in the active state 220. For example, the FSM 200 may disable any of the low fidelity sensors 150 and enable and/or increase the max fidelity settings of any high fidelity sensors 155.

Further tasks may be performed during the active state 220, for example, the EMM 110 may also detect and handle input and output operations, periodically wake to take a data point, or perform any other additional active state tasks 275 as required or desired. In one embodiment, data points may be spaced at a regular period (e.g., minute(s) intervals) to capture data throughout the time the user is using the ISPM 100. In other embodiments, data points may be spaced seconds apart to generate higher resolution activity data such as sprinting acceleration or deceleration. Some embodiments may count the certain number of events over a time duration called an event count. EMM 110 may asynchronously tally events (e.g., steps or hits) over a time duration. After the time duration has expired, the total count and time duration are recorded, providing rate information in terms of events per time period (e.g., step count or hit count). After recording the data point, the tally count is reset and the process continues. Any duration may be chosen and modulated as needed. In other embodiments, data points may be taken after a certain number of events are tallied. For example, EMM 110 may asynchronously tally events (e.g., steps or hits). Upon reaching a number of events threshold (e.g., ten steps), a data point may be taken recording the number of events and the duration, providing rate information in terms of events per duration (e.g., step count or hit count). The number of events threshold may be modulated. In general, event counts (e.g., step count or hit count) provide event tallies during a time duration. Any sensor configured to generate an interrupt may take data points as event counts instead of, or in addition to, polled data. Methods of taking data points may be combined and used as needed by the specific application. Data points may be generated at non-periodic intervals as required by the application. Upon completing additional active state tasks 275, the FSM 200 may return back to active state 220. While in the active state 220, if no activity is detected over a longer duration than a programmable timeout period, the FSM 200 may revert back to the idle state 205 via idle state reversion 265 where the EMM 110 may reconfigure to the previously used sensor configurations and tasks of the idle state 205.

Continuing with FIG. 9, the capture state 230 is the FSM 200 state optimized for high fidelity data capture. As discussed above, during the capture state 230, the low fidelity sensor 150 may be disabled or set to low power settings, whereas the high fidelity sensor may be enabled and/or throttled up to max fidelity. During the capture state 230, the EMM 110 records the remainder of the target event (after detection of the capture trigger portion of said target event) at full data fidelity. The high fidelity sensor 155 and any additional desirable sensors for data capture are enabled and data rates throttled up to the fidelity required by the application. The capture state 230 represents the highest power mode, however the total burst duration lasts a relatively short period of time. For example, in an embodiment, 20 ms may be sufficient to record the full concussive level head impact profile. The sampling period may be on the order of milliseconds, providing the highest resolution data at the instant of impact. The sensor data may be recorded in memory or directly transmitted through an output device 165 or bi-communication module 160 (as illustrated in FIG. 7). When the memories are filled or a timeout period elapses the FSM 200 transitions to the decision state 240 as represented by decision state transition 235. Decision state transition 235 is a transitional period in which the FSM 200 may alter sensor configurations, disable sensors, and/or generally perform new tasks in addition to or to the exclusion of the tasks performed in the capture state 230. For example, the FSM 200 may disable any or all of the low fidelity sensors 150 and/or high fidelity sensors 155 to conserve power.

Continuing with FIG. 9, the decision state 240, is an optional processing of the capture state 230 data, which may confirm or reject a target event as being potentially injurious (or other designation as desired) and potentially issue an alert if confirmed (e.g., via output device 165). The initiation of said target event may have been recognized by sensors in the active state 220 as the capture trigger. As discussed above, detection of the capture trigger results in high fidelity measurement in the capture state 230, and the generated data is processed in the decision state 240. The decision state 240 may use the recorded data from the capture state 230. An algorithm may be used to categorize the recorded data into a confirmed event (e.g., a THI) or a false positive. The Head Injury Criterion (HIC), or HIC formula, is an example algorithm used to score recorded head accelerations with respect to a head injury probability index. Further examples of algorithms which may be used in the disclosed embodiments may be found in U.S. patent application Ser. No. 13/934,432 the disclosure of which is incorporated herein by reference. The HIC formula may be used to classify a target event as a confirmed event or a false positive. The HIC formula is illustrated as equation 1 below:

$$HIC = {\{ {[ \frac{1}{{t}_{2} - {t}_{1}} {\int }_{{t}_{1}}^{{t}_{2}} a ( t ) d t ]}^{2.5} ( {t}_{2} - {t}_{1} ) \}}_{ma x} \qquad ( Eq . 1 )$$

Typically, the HIC is derived from the acceleration/time history of an accelerometer mounted at the center of gravity of a dummy's head when the dummy is exposed to crash forces. In the equation, t₁ and t₂ are the initial and final times (in seconds) of the interval during which HIC attains a maximum value, and acceleration a is measured in gs (standard gravity acceleration). The maximum time duration of HIC, t₂−t₁, may be limited to a specific value between 3 and 36 ms. An HIC score exceeding a programmable level may be defined as a confirmed event, whereas a score below the programmable level may be defined as a false positive. If a false positive is detected the FSM 200 reverts back to the active state 220 via active state reversion 255. Conversely, a confirmed event transitions the FSM 200 to the notification state 250 via notification state transition 245. The HIC is only an example of a calculation made by the decision state 240. Different applications may necessitate different computational models. The result determined by the decision state 240 may be recorded in memory or directly transmitted through an output device 165 or bi-communication module 160 as an alert. In some applications, a capture trigger may automatically result in a detected event should the capture trigger reach a preprogrammed threshold or should the FSM 200 be programmed as such. In these, as well as other embodiments, the decision state 240 may be optional. Additional algorithms may be used in place of, or in addition to the HIC, including peak acceleration, Gadd severity index (GSI), and others as would occur to one having ordinary skill in the art. Categorization of confirmed events or a false positives may vary on an individual basis. For example, metrics such as gender, age, height, weight, injury history, medical history, and position may factor into the categorization. As discussed above, if a target event is confirmed by the analysis performed in the decision state 240, the FSM 200 transitions to the notification state as represented by notification state transition 245. Notification state transition 245 is a transitional period in which the FSM 200 wherein the FSM 200 may generally perform new tasks in addition to or to the exclusion of the tasks performed in the decision state 240. Generally, the FSM 200 may maintain any disabled sensors in the disabled or low power states as they were in the decision state 240 and/or the FSM 200 may disable sensors that were not disabled in the decision state 240. Alternatively, should a false positive be detected, the FSM 200 may revert back to active state 220 via active state reversion 225. During active state reversion 225 the FSM may alter the sensor configurations and tasks performed to those previously used during active state 220 or it may alter such configurations and tasks based on any analysis of the false positive, such that it may use new sensor configurations and/or tasks to reduce the incidence of potential false positives.

The notification state 250 defines the period when the EMM 110 alerts the user or apparatus to the confirmed event. The alert may be performed by an output device 165 or bi-directional communication module 160. In embodiments where the output device 165 is a visual indicator, an LED may be illuminated or pulsed to signal an event. In embodiments where the output device 165 is an RF transmitter, a radio signal may be an event. The alert carried out by the notification state 250 may be recorded in memory in addition to, or as an alternative to indicating the alert by output device 165 or bi-directional communication module 160. The notification state may be an optional state. After a timeout period, FSM 200 may revert back to the active state via active state reversion 255. During active state reversion 225 the FSM may alter the sensor configurations and tasks performed to those previously used during active state 220 or it may alter such configurations and tasks as desired. The alert may indicate to an athlete or administrator the need to perform a correction in real-time.

Though details of the FSM 200 are enumerated above, it is to be understood that modifications and additional features may be incorporated into the FSM 200 as desired. Additional FSM states may be added. For example, a secondary active state may be added between existing active state 220 and capture state 225 to provide for additional levels of power management. Conversely, FSM states may be removed. For example, the active state 220 may be completely eliminated such that the FSM transitions directly from idle state 205 to capture state 230.

The data recorded by the FSM 200 and produced by the EMM 110 may be characterized by non-uniformly spaced data points. As the rate of change of the desired signal increases, the system may automatically adjust the sampling rate and resolution to compensate. When high sampling rates and resolutions are not needed, the EMM 110 does not collect data using high sampling rates or resolutions. In some embodiments, data may not be collected at all when there is no trigger (e.g., activity trigger) to do so. As a result, data points are naturally and efficiently clustered around regions of interest, while sparsely populating regions of little interest.

Embodiments of EMM 110 may comprise a Companion Sensor System (CSS), examples of companion sensors, primary sensors, and companion sensor systems are described in U.S. patent application Ser. No. 13/934,432 the disclosure of which is incorporated herein by reference. The low fidelity sensor 150 may function as the companion sensor. For example, the low fidelity sensor 150 may generate binary data with such data being used as an activity trigger. The low fidelity sensor 150 may comprise a passive sensor featuring low or no power requirements to operate as a companion sensor. A high fidelity sensor 155 may function as a primary sensor. As the low fidelity sensor 150 (i.e. the companion sensor) modulates the sampling rate of the high fidelity sensor 155 (i.e. the primary sensor), the low fidelity sensor 150 modulation determines the rate of data sampling of the high fidelity sensor 155. Therefore, the low fidelity sensor 150 regulates the frequency and time with which data collection occurs. In some embodiments, the EMM 110 settings may be adjusted by the user.

The user may adjust the system event and alert settings of the EMM 110 by either wired connection or through a wireless connection. An example of an alert modification might be a HIC score magnitude notification using an output device 165. For example, a user may set which HIC value or range of values corresponds with a given LED color, LED flash rate, audio tone, audio beep, vibration intensity, or vibration buzz rate, for example an HIC score of 50 to 79 may use a yellow illumination, whereas an HIC score greater than 80 may use a red illumination. Alternatively, a cumulative measure of HIC scores may be used. A user may preprogram the number of significant (which would be determined by the user for a given application) HIC scores needed for a given health alert. For example, three significant HIC scores may use a yellow illumination, whereas the fourth significant HIC score would use a red illumination. Alternatively, alerts may be modulated according to time or level of activity. An example may be to alert the user to a change in behavior during an athletic activity, for example, running faster or slower during a workout. Further, the user may adjust the activity trigger and capture trigger thresholds, as well as the idle state 205, active state 220, and capture state 230 sampling rates and dynamic ranges. In addition to the type of alert, the duration of the alert may also be programmable. As discussed above, the algorithm used during the decision state 240 may be adjusted. Other settings may be programmable as may occur to one skilled in the art. Further, the settings of the EMM 110 may be tuned on a per person basis, per team basis, or set based on heuristics derived from previously gathered data sets. For example, a collection of data on head injuries correlated with demographic parameters such as gender, age, weight, height, position may be used with the EMM 110 to provide optimized settings on an individual specific level.

The EMM 110 may be configured to simultaneously record multiple kinds of data at differing data rates. For example, the wearer's step count may be recorded by logging every acceleration exceeding a step threshold during the active state 220, or a hit count may be recorded by logging every acceleration that trips the capture trigger threshold regardless of the output of the decision state 240. Other data including temperature, battery voltage level, humidity, pulse oximeter reading, sprint acceleration, etc. may be recorded as well.

In some embodiments, the EMM 110 may include GPS capability. An example of an EMM 110 with GPS capability comprises pairing data recorded by EMM 110 with a map application. The distance and rate of travel during EMM 110 use may be recorded. Color coded routes may be indicated on a map application to allow user correction. GPS information may be entered by the user either before use or in situ by direct connection of the EMM 110 or via wireless communication. The EMM 110 may alert the user of a correction by either a visual signal (e.g., LED), an audio signal, or a tactile (vibration) signal.

Input and output information from the EMM 110 may be relayed through an auxiliary relay device (“ARD”). An ARD may be placed in standard athletic equipment including but not limited to headbands, skullcaps, helmets, goggles, facemasks, hair ties, swim caps, pads/shin guards, gloves, wristbands, anklebands, shoes, socks, ice skates, bra, oral appliance, or chest straps. The ARD may also be placed in equipment that is not wearable or may be located at locations separate from the EMM 110. For example, the ARD may be located on the sideline of an athletic field while the EMM 110 is located in the helmet of an athlete on said athletic field. The ARD may comprise a battery, processor, an input device, an output device, primary sensors, companion sensors, etc. The ARD may communicate with one or more EMMs 110 or with additional ARDs. Further examples of ARD's may be found in U.S. patent application Ser. No. 14/076,849 the disclosure of which is incorporated herein by reference.

In some embodiments, EMM 110 may provide for a time calibration source such that the generated data may be time synchronized to other data sources. Two requirements may be met; the EMM 110 may establish a precise oscillator and the EMM 110 may be provided at least one external time stamp reference point. The oscillator need not be accurate provided the EMM 110 is able to calibrate said oscillator source. For example, a processor 140 may include a precise low power RC oscillator for time keeping, and an accurate factory calibrated oscillator for the system clock. The system clock may be used as a reference to calibrate the low power RC oscillator. Alternatively, both accuracy and precision may be established including an external oscillator, such as a 32.768 kHz crystal oscillator. Bi-directional communication module 160 may provide one or more absolute time points to the EMM 110. Combining the absolute time points with a precise oscillator enables EMM 110 data to be synchronized in time with other data sources.

In some embodiments, the EMM 110 may synchronize data with multiple data sources, such as multiple ISPMs, ARDs, GPS, video, or other data generated in concert with the EMM 110. For example, in some applications, it may be desirable for a team using multiple ISPMs 100 to record synchronized data. As a result, the data generated by each user may be readily compared in time to any other user. In other embodiments, EMM 110 data may be synchronized to a video recording. For example, a team of athletes may wear ISPMs 100 while being recorded on video. EMM 110 may be synchronized in time with the video recording. Users may view, in real-time or post game, the combination of video recording with EMM 110 data, providing additional insight on activity, events, alerts, and corrections. Continuing the example, the EMM 110 may indicate the time of a potential THI. In a post game review, the time information may enable viewing EMM 110 data and video footage simultaneously, providing additional health and performance insight. Additionally, EMM 110 alerts may automatically annotate video or other data sources in real-time or for post game review.

In some embodiments, the EMM 110 may provision for automatic calibration of any of the low fidelity sensors 150 and/or high fidelity 155 sensors. In some embodiments, the EMM 110 may comprise at least two discrete sensors designed for different applications, therefore, in some applications; the sensors may be able to automatically calibrate each other. For example, a low fidelity sensor 150 and high fidelity sensor 155 may be able to calibrate against each other. As a specific example, two accelerometers having different power modes, dynamic ranges, resolutions, and/or offset errors may be used to directly calibrate the offset error in one or both of the accelerometers. This process may allow for field re-calibration in any orientation.

In some embodiments, the EMM 110 may incorporate a built-in balance tester. In applications such as head injury recognition, it may be desirable to directly measure an athlete's balance ability. The EMM 110 may be configured to perform a balance assessment before and after a potential injury. Changes in the balance assessment results may be used as additional indicators of potential head injuries requiring medical assistance.

In a specific application, the ISPM 100 may be used as a soccer on-field concussion management device. The BAC 105 may take the form of a headband as shown in FIGS. 1 and 2. The size and shape of the EMM 110 may be minimized to be nominally hidden. The EMM 110 may comprise a soft rubberized bumper on its battery door 125 and top housing 135 to improve comfort and safety. The EMM 110 may comprise at least two accelerometers. The low fidelity accelerometer 150 may be configured to detect running signatures as an activity trigger. Upon detection of the activity trigger, the low fidelity accelerometer 150 may trigger the processor 140 to change the FSM 200 to the active state 220 via active state transition 210. In the active state 220, the high fidelity accelerometer 155 may detect a capture trigger. The activity trigger may designate the onset of a potentially dangerous head movement that may result in a THI. The activity trigger threshold level may be specifically tailored to the wearer. Upon detection of the capture trigger, the FSM 200 may transition to the capture state 230 via capture state transition 225. In the capture state 230, the high fidelity sensor 155 may be reconfigured to record the target event (e.g., a head impact profile), at high data fidelity settings. Once sufficient data has been captured, the FSM 200 may transition to the decision state 240, and the high fidelity sensor 155 may be disabled. An algorithm (e.g., the HIC score) may be computed and compared against a user programmable threshold. Exceeding a threshold individualized to the athlete results in a confirmed event the FSM 200 may transition to the notification state 250 via notification state transition 245. In the notification state 250, an output device 165 may notify a user or apparatus of by issuing an alert (e.g., an LED). After a programmable timeout elapses, FSM 200 may resume execution in the active state 220 via active state reversion 255. Further, the EMM 110 may record and track a player's step count, hit count, threshold exceeding hits, and sprint accelerations to quantify changes in the player's health and game performance throughout a season. When the ISPM 100 is no longer in use, timeout idle state reversion 265 may elapse placing the EMM 110 into the idle state 205 where it operates in its lowest power mode for maximum longevity. The player need not turn off the ISPM 100 as the FSM 200 manages power usage autonomously. The ISPM 100 may enable the EMM 110 to perform for an entire season on a single charge, thus minimizing the inconvenience of recharging and remembering to turn on and off the device between uses. The present embodiments may dramatically reduce the cost associated with on-field concussion recognition and concussive hits may be detected more reliably with the final result being a reduction in the frequency of SIS. It is to be understood, however, that the ISPM 100 is not to be limited to a specific activity. The ISPM may be used with any activity that carries a risk of a collision including athletic activities, workplace activities, healthcare activities, etc. Examples of activities include but are not limited to: soccer, football, lacrosse, track and field, hockey, gymnastics, acrobatics, diving, figure skating, volleyball, ski, motor sports, skate board, cycling, construction work, military activities; activities involving geriatrics, infants and children, the mentally ill; patients at risk for falls, seizures, etc.

As discussed above, embodiments may be used in applications beyond detection of THI. The capture trigger threshold may be changed and/or different sensors or sensor configurations may be used to measure data from numerous applications. For example, a head mounted ISPM 100 may be used to assess the performance of a soccer player's playing ability, specifically the player's abilities of heading the ball. Upon heading the ball, the full details of the accelerations involved in the header may be transmitted live to a coach or trainer via a portable tablet such that the real-time evaluation may be possible. In some applications, a soccer coach may be interested in the strength or form of a player's heading ability. The ISPM 100 may provide the actionable data to improve athlete performance. Other applications may include quantifying the speed and power of a football player blocking an opposing team player. The EMM 110 may be secured to the hands of the player via a glove (as shown in FIG. 4). The activity trigger and capture trigger thresholds may be modified to the necessary levels. Then upon detection of a block, the data may be transmitted live to a coach or trainer via a portable tablet such that the real-time evaluation may be possible. Further applications may include smart gloves (as shown in FIG. 4) for soccer goalies. A single low fidelity sensor 150 may be used to detect an activity trigger. During the capture state 230, a plurality of high fidelity sensors 155 located on the fingers of the gloves may be enabled to help quantify the ball handling ability of a goalie. Applications in mixed martial arts may comprise a sensor configured to track punches, kicks, and head hits. Multiple ISPMs 100 may be placed about the athlete, including head, torso, feet, and fists, and used in parallel for a complete assessment of physical activity. Yet other applications include tracking for track and field athletes. For example, the EMM 110 may be attached to the body by either a headband (as shown in FIGS. 1 and 2) or a wristband. For this application, the desired output data may simply include the duration and intensity of running. The capture state 230 and capture trigger may be eliminated. When the ISPM 100 is used, the EMM 110 enters the active state 220 and records the intensity and duration of the work-out regime. Additional modifications to the FSM 200 may include an indicator which notifies the player to change their activity, for example, to speed up, slow down, change run type, etc. The indicator may be based on a pre-programmed training regimen. The actual results of the regimen may be compared against the desired program by the coach either offline or in real-time.

### EXAMPLES

To facilitate a better understanding of the present claims, the following examples of certain aspects of the disclosure are given. In no way should the following examples be read to limit, or define, the entire scope of the claims.

### Example 1

Example 1 is a comparative example used to compare the properties of an example low fidelity sensor and high fidelity sensor individually against the properties of the sensors used in combination, for example, in the EMM 110. Two commercially used accelerometers, were compared. The first accelerometer is the LIS2DH, which is an example of a low fidelity sensor 150. LIS2DH is commercially available from STMicroelectronics of Geneva, Canton of Geneva. The second accelerometer is the ADXL375, which is an example of a high fidelity sensor 155. ADXL375 is commercially available from Analog Devices, Inc. of Cambridge, Massachusetts. As illustrated by Table 1, the max dynamic range of the LIS2DH is ±16 gs with down to 8-bit resolution. The LIS2DH comprises a power mode of operation of 4 uA consumption at a 25 Hz sampling rate. Conversely, the ADXL375 comprises a 16-bit resolution and a dynamic range up to ±200 gs. In capture state 230 operation, the ADXL375 consumes 140 uA, and in idle state, consumes 40 uA. Both sensors sample at a constant rate.

TABLE 1
Comparative Example

| Metric | LIS2DH | ADXL375 | EMM |
|---|---|---|---|
| Max dynamic range | ±16 gs | ±200 gs | ±200 gs |
| Resolution | 8 bit | 16 bit | 16 bit |
| Standy-by/Power-down Current | 0.5 uA | 0.1 uA | NA |
| Consumption | | | |
| Idle State Current Consumption | 4 uA | 40 uA | 4 uA |
| (25 Hz) | | | |
| Active State Current Consumption | 36 uA | 90 uA | 36 uA |
| (400 Hz) | | | |
| Capture State Current Consumption | Not | 140 uA | 140 uA |
| (800 Hz) | Applicable | | |
| Sampling Rate | Constant | Constant | Dynamic |

In the current example, a minimum of ±200 gs may be required for detection of a potential THI. Thus, an EMM 110 would require the ADXL375 with a relatively high power consumption of 40 uA in the idle state 205. Due to the high power consumption, the design may require the user to perform invasive operations such as manually turning the device on or off between uses or requiring frequent recharging. These interactions decrease user engagement and compliance over time. Additionally, larger batteries may also be required to the discomfort of the wearer.

As illustrated by Table 1, using the two sensor types together may dramatically decrease power consumption by an order of magnitude during the idle state by interleaving use of the LIS2DH and the ADXL375 and modulating sampling rates. During idle state 301, the ADXL375 may be shut down (consuming only 0.1 uA) and the LIS2DH enabled, consuming 4 uA. Only when the activity trigger is detected, the high fidelity sensor ADXL375 may be enabled and sampling rate increased. Next, upon detecting the capture trigger, the ADXL375 sampling rate may be increased to measure the full head acceleration profile at full fidelity settings during the capture state 230. Though current consumption in capture state 230 exceeds 140 uA, the FSM only stays in this state for a short duration of ˜20 ms and then enters the decision state where the ADXL375 is fully disabled and resumes a current consumption of 0.1 uA. The increased data rate during capture state 230 clusters data points around areas of interest, dramatically improving the data gather efficiency of the EMM 110. The efficiency is improved because the EMM samples sparingly during periods of little or no activity and densely samples during regions of known interest. Only after two pre-requisite trigger events (e.g., an activity trigger and a capture trigger), does the EMM sample at maximum data rate but also at maximum power. The sensor spends the vast majority of time in idle state 205 (on the order of weeks), less time in active state 220 (on the order of hours), and even less during capture (on the order of milliseconds). The composite effect enables the EMM 110 to achieve more than what either sensor could achieve alone, ultra-low power, high-fidelity data capture, and high efficiency in data point clustering. The result is a dramatic change in the way users interact and how long they are likely to continue interacting with wearable monitors.

As seen in the LIS2DH and ADXL375 example, a plurality of sensors and data rates may be mapped across the FSM 200 states in order to optimize power consumption while maintaining high-fidelity data capture. However, using the same principles, the FSM may additionally map across the sensor's built-in power modes to further optimize longevity. During the active state 220, the LIS2DH sampling rate may be increased to match nominal sampling rate of 400 Hz without enabling the ADXL375. In this mode, the EMM 110 continues to benefit from an additional 3× improvement in active time longevity. Upon detecting an acceleration exceeding a level within its dynamic range of +16 gs, the FSM 200 transitions to the capture state 230 and the ADXL375 is turned-on at the moment when the measurement needs to be taken. This technique works when the turn-on time of the secondary sensor is significantly shorter than the bandwidth of the signal to be measured. In this case, the turn-on time of the ADXL375 is negligible compared to the acceleration profile of a head impacting a ball. Additional sensors such as a gyro may also be enabled during the capture state 230 if the turn-on time is less than the signal bandwidth.

Example embodiments were chosen and described in order to best explain the principles of the invention and their practical application, and to enable others of ordinary skill in the art to understand the invention for various embodiments with various modifications as are suited to the particular use contemplated. Those skilled in the art will understand, however, that the invention may be embodied as many other devices, systems, and methods. For example, various aspects of the methods and devices may be combined in various ways or with various products, including existing products. Many modifications and variations will be apparent to those of ordinary skill in the art. To the extent such modifications fall within the scope of the appended claims and their equivalents, they are intended to be covered by this patent. The scope of the invention is not intended to be limited by the details of example embodiments described herein. The scope of the invention should be determined through study of the appended claims. Although individual embodiments are discussed herein, the invention covers all combinations of all those embodiments.

1. An in-situ physiologic monitor for a body comprising:
an electronic monitoring module, wherein the electronic monitoring module is capable of logging an electronic monitoring module sensed parameter; and
a body attachment component, wherein the body attachment component attaches the electronic monitoring module to the body.

2. The in-situ physiologic monitor of claim 1, wherein the electronic monitoring module comprises a low fidelity sensor and a high fidelity sensor, wherein the low fidelity sensor consumes less power than the high fidelity sensor, wherein the high fidelity sensor is capable of sensing in a higher fidelity than the low fidelity sensor.

3. The in-situ physiologic monitor of claim 2, wherein the low fidelity sensor comprises an accelerometer, magnetic sensor, reed relay, RFID, photo-sensor, microphone, pressure sensor, piezoelectric sensor, tilt sensor, radio antenna, or combination thereof.

4. The in-situ physiologic monitor of claim 2, wherein the high fidelity sensor comprises a thermocouple, pressure sensor, accelerometer, electro-chemical sensor, gyroscope, humidity sensor, microphone, acoustic sensor, vibration sensor, temperature sensor, hydration sensor, humidity sensor, moisture sensor, proximity sensor, light sensor, tilt sensor, inertial measurement unit sensor, compass, inclinometer, altimeter, GPS, pulse-oximeter sensors, EEG, EKG, voltmeter, ammeter, capacitance meter, inductance meter, resistance meter, LCR meter, or any combination thereof.

5. The in-situ physiologic monitor of claim 1, wherein the body attachment component comprises headband, helmet, skull cap, goggles, facemask, hair tie, swim cap, shin guard, elbow pad, knee pad, glove, wristband, ankleband, shoulder pad, shoes, socks, ice skates, bra, jersey, oral appliance, clip, cheer bow, or chest strap.

6. The method of claim 1, wherein the electronic monitoring module sensed parameter comprises an acceleration, rotation, tilt, displacement, velocity, position, orientation, step count, hit count, HIC score, hits per epoch heart rate, temperature, distance, time, calories burned, speed, sprint acceleration, hydration, balance, reaction time, voltage, or combinations thereof of the electronic monitoring module

7. The in-situ physiologic monitor of claim 6, wherein the electronic monitoring module further comprises an output device configured to provide visual feedback, haptic feedback, tactile feedback, audible feedback, radio frequency feedback, or a combination thereof.

8. The in-situ physiologic monitor of claim 1, wherein the body attachment component comprises an enclosure in which the electronic monitoring module is disposed.

9. A method of conserving power in an in-situ physiologic monitor for a body, the method comprising:
providing an in-situ physiologic monitor comprising:
an electronic monitoring module; wherein the electronic monitoring module is capable of logging one or more electronic monitoring module sensed parameters; wherein the electronic monitoring module comprises a low fidelity sensor and a high fidelity sensor; wherein the low fidelity sensor consumes less power than the high fidelity sensor; and wherein the high fidelity sensor is capable of sensing in a higher fidelity than the low fidelity sensor; and
a body attachment component, wherein the body attachment component attaches the electronic monitoring module to the body; and
using the low fidelity sensor to induce sensing with the high fidelity sensor.

10. The method of claim 9, wherein the low fidelity sensor comprises an accelerometer, magnetic sensor, reed relay, RFID, photo-sensor, microphone, pressure sensor, piezoelectric sensor, tilt sensor, radio antenna, or any combination thereof.

11. The method of claim 9, wherein the high fidelity sensor comprises a thermocouple, pressure sensor, accelerometer, electro-chemical sensor, gyroscope, humidity sensor, microphone, acoustic sensor, vibration sensor, temperature sensor, hydration sensor, humidity sensor, moisture sensor, proximity sensor, light sensor, tilt sensor, inertial measurement unit sensor, compass, inclinometer, altimeter, GPS, pulse-oximeter sensors, EEG, EKG, voltmeter, ammeter, capacitance meter, inductance meter, resistance meter, LCR meter, or any combination thereof.

12. The method of claim 9, wherein the electronic monitoring monitor further comprises an idle state, an active state, and a capture state.

13. The method of claim 12, wherein when the electronic monitoring module is in the idle state, the low fidelity sensor is sensing and the high fidelity sensor is not sensing.

14. The method of claim 12, wherein when the electronic monitoring module is in the active state, the low fidelity sensor is sensing and the high fidelity sensor is sensing.

15. The method of claim 12, wherein when the electronic monitoring module is in the capture state, the low fidelity sensor is not sensing and the high fidelity sensor is sensing.

16. The method of claim 12, wherein the electronic monitoring module sensed parameter comprises an acceleration, rotation, tilt, displacement, velocity, position, orientation, step count, hit count, HIC score, hits per epoch heart rate, temperature, distance, time, calories burned, speed, sprint acceleration, hydration, balance, reaction time, voltage, or combinations thereof of the electronic monitoring module

17. The method of claim 10, wherein the inducement of sensing with the high fidelity sensor further comprises:
using the low fidelity sensor to detect an electronic monitoring module sensed parameter above a preset threshold,
enabling the high fidelity sensor and/or throttling up the fidelity rate of the high fidelity sensor, and
capturing data from the electronic monitoring module sensed parameter in high fidelity.

18. The method of claim 17, further comprising the electronic monitoring module throttling the fidelity rate of the high fidelity sensor down or disabling the high fidelity sensor after capturing the data from the electronic monitoring module sensed parameter.

19. The method of claim 17 further comprising analyzing the captured data from the electronic monitoring module sensed parameter.

20. The method of claim 19, wherein the electronic monitoring module further comprises an output device; wherein the output device provides a feedback selected from the group consisting of visual feedback, haptic feedback, tactile feedback, audible feedback, radio frequency feedback, and a combination thereof; and wherein the feedback is provided in response to the analyzed capture data.
